(19) United States
(12) Patent Application Publication
(10) Pub. No.: US 2003/0093002 A1
(43) Pub. Date: May 15, 2003

(54) Function indicator for autonomic nervous system based on phonocardiogram

(76) Inventor: Terry B.J. Kuo, Hualien Hsien (TW)

Correspondence Address: J.C. Patents, Inc., 4 Venture, Suite 250, Irvine, CA, 92618, US

(21) Appl. No.: 09/990,992

(22) Filed: Nov. 13, 2001

(51) Int. Cl. A61B 5/02

(52) U.S. Cl. 600/528

(57) ABSTRACT

A method and an apparatus for measuring the heart rate variability (HRV) are described. A recording of the heart sounds is processed and analyzed with a computing system to obtain various components that characterize the heart rate variability. Since changes of HRV are derived from the sound signals of a heart, which is readily collectable with a microphone or a listening instrument used in auscultation and is readily accessible to patients, a rapid diagnosis and transfer of information are provided. Potential consequences are curtailed and the survivability of patents is thereby enhanced.

### BACKGROUNDING OF THE INVENTION

1. Field of Invention

The present invention relates to a method and an apparatus for monitoring the autonomic nervous system. More particularly, the present invention relates to a method and an apparatus for measuring the heart rate variability (HRV) based on a recording of heart sounds (phonocardiogram).

2. Description of Related Art

The autonomic nervous system (ANS) regulates individual organ function and homeostasis, such as heart beat, digestion, breathing and blood flow, and for the most part is not subject to voluntary control. These involuntary actions are controlled by the opposite actions of the two divisions of the autonomic nervous system—the sympathetic and the parasympathetic divisions. Most organs receive impulses from both divisions and under normal circumstances and they work together for proper organ function and adaptation to the demands of life. Problems will occur when the autonomic nervous system is out of balance, for example, coronary heart disease, hypertension, digestive disturbances and even sudden death.

Many techniques have been successfully developed to assess the autonomic nervous system. These techniques include heart rate variation with deep breathing, Valsalva response, sudomotor function, orthostatic blood pressure recordings, cold pressor test and biochemistry test, etc. These techniques, however, are mostly invasive and employ expensive diagnostic instruments. These techniques are, therefore, not appropriate for general applications.

Many believe that patterns of heart rate variation relate closely to the modulation of the autonomic nervous system. Heart rate variability (HRV) has been developed as a function indicator of the autonomic nervous system. HRV refers to the beat-to-beat alterations in the heart rate. It is a measure of the beat-to-beat time interval variations as the heart speeds up or slows down with each breath under a precordial state. Among the different techniques in assessing the autonomic nervous system, HRV is an important breakthrough because this technique is non-invasive. Moreover, the hardware for the technique is inexpensive, and thus can broadly apply. In addition, animal and clinical studies confirm HRV accurately reflects the sympathetic and parasympathetic activities and their balance.

In adult at rest there is about 70 heart beats per minute. These rhythmic heart beats are originated from an electrical event coupling between cardiac muscle cells which include the myocardial cells, the nodal cells and the conducting cells. The heart receives impulses from both the sympathetic and the parasympathetic divisions of the autonomic nervous system, which normally work together for a proper functioning. However, if the body is stressed, the sympathetic nervous system dominates causing an increase in heart rate and blood pressure. When the emergency situation has passed the parasympathetic system takes over and decreases the heart rate. The maintenance of the heart rate further includes many frequent and detailed neurological controls, which involve intricate dynamic feedback mechanisms. The heart rate of a healthy individual thereby exhibits minor periodic variations, which occur every ten seconds or every three seconds.

Recent developments in electrical engineering have enabled the assessment of heart rate variability by frequency domain analysis, which bases on mathematical manipulations performed on the ECG-derived data. FIG. 1 is a flow diagram illustrating the conventional approach in assessing heart rate variability. As shown in FIG. 1, an electricocaridogram (ECG) is first taken from a subject. The ECG signals are then amplified, filtered and digitized. A computer program for HRV analysis is then used to process the ECG signals. The computer first detected all peaks of the digitized ECG signals. The interval between two peaks is then estimated. The frequency-domain measurements are further quantified by using a nonparametric method of fast Fourier transform (FFT).

Investigators have discovered that, based on frequency analysis, HRV can be characterized into two main components: the high frequency (HF) component and the low frequency (LF) component. The high frequency component is equivalent to respiratory sinus arrhythmia and is considered to represent the influence of the vagal control of the heart rate. The exact origin of the low frequency component is not known. It is probably related to vessel activity or baroreflex. Some investigators further divide the low frequency component into a low frequency component and a very low frequency component.

It is well documented that HRV is clinically valid and meaningful in reflecting many physiological functions. Many investigators discover that the high frequency component or the total power (TP) can consider representing the parasymapthetic control of the heart rate and the ratio LF/HF is considered to mirror the sympathovagal balance or to reflect the sympathetic modulations. Reduced HRV appears to be a marker of an increase of intra-cranial pressure. Lowered HRV is also shown to associate with aging. HF has been shown to decrease in diabetic neuropathy, whereas LF/HF is sensitive to postural change and metal distress. In a human study, LF is shown to be eliminated in brain death and can be used as a prognostic tool for the prediction of patent outcome in the intensive care unit. A recent study by Framingham further indicates that if the HRV of an elderly is lowered by one standard deviation, the HRV of a near-death individual is about 1.7 times lower than a normal individual.

Although HRV is a promising in predicting various pathological states, it is a measurement that still has unresolved issues. While the periodic variation of the heart rate, determined by means of the frequency analysis of an ECG signal, can be used to provide us with an indirect assessment of the autonomic nervous system, the acquisition of an ECG signal is not convenient to accomplish. In order to obtain information on HRV, patents need to use an ECG module, in which the beat-to-beat interval in heart rate can be derived and from which the variation in heart rate can be measured. The acquisition of an electrocardio signal further requires a proper placing of a plurality of electrodes on various parts of the body.

Since changes of HRV occur in response to many common yet deadly diseases, such as coronary heart disease and hypertension, having a method and an apparatus that is readily accessible to patents, and can provide a rapid diagnosis and transfer of information would curtail potential consequences and thus enhance the survivability of patents.

### SUMMARY OF THE INVENTION

Accordingly, the present invention provides a method and an apparatus for monitoring the autonomic nervous system by measuring heart rate variability (HRV), wherein signals of the heart beat are more convenient and readily to access.

Accordingly, the present invention provides an apparatus for monitoring the heart rate variability, wherein the apparatus is easy to operate, can be portable and be used at the convenience of the user. The apparatus for measuring HRV of the present invention includes a microphone to collect the sound signals of a heart. The apparatus further comprises an amplifier, a filter and an analog-to-digital converter to process and to digitize the sound signals. The apparatus also comprises a computer for analyzing the sound signals and generating meaningful physiological and clinical results. The analyzed results can be viewed on-line by the user during the test or sent to other computer systems for an off-line verification after the completion of the test.

The present invention provides a method for measuring the HRV of a subject, wherein a microphone is placed near the heart of the subject to collect three to five minutes of the sound signals of the heart. The sound signals of the heart are amplified, filtered and transmitted to an analogy-to-digital (A/D) converter.

The digitized sound signal is then analyzed to determine the beat-to-beat interval using a computer. Parameters such as amplitude and duration of all peaks are determined so that their means and standard deviations are calculated as standard templates. Each subsequent heart rate is then compared with the standard templates. The power spectral density is further estimated on the basis of fast Fourier transform and is subsequently quantified by means of integration into standard frequency-domain measurements including low frequency (LF), high frequency (HF), total power and LF/HF. Then these parameters are logarithmically transformed.

Since the HRV of the present invention is derived from a phonocardiogram, which is easily obtained by placing a microphone on a patient, the pathological conditions of a patent is readily assessable and diagnosed. Moreover, the phonocardiogram and the corresponding HRV information even they are collected at the patient's own home can be sent to computer systems for an off-line verification after the completion of the test. With a rapid diagnosis and transfer of information, potential consequences are mitigated and the survivability of a patient is enhanced.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a flow diagram illustrating the conventional approach in assessing heart rate variability;

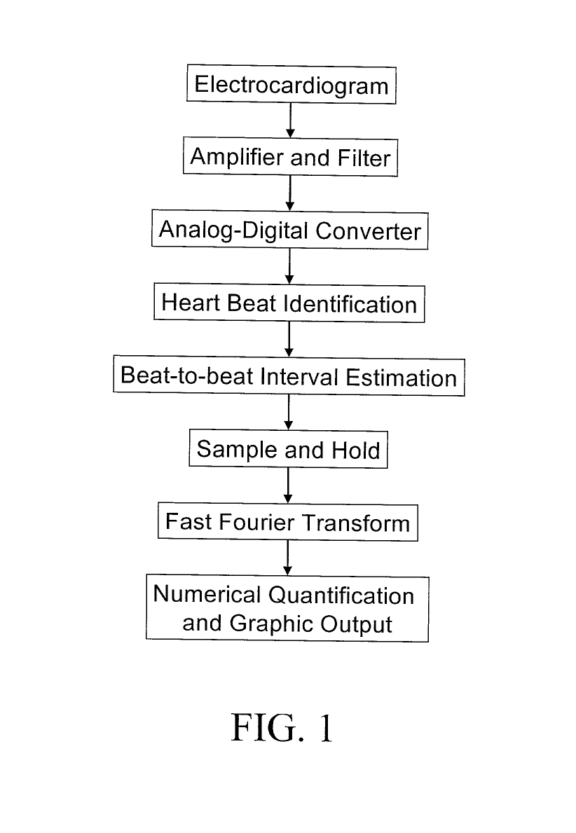

FIG. 2 is a flow diagram illustrating the approach in assessing heart rate variability according to the present invention;

FIG. 3 illustrates a phonocardiogram and the corresponding beat-to-beat intervals of a five-minute study on a subject according to the method of the present invention. The dots indicate the peaks of the heart rate automatically identified by a computer;

FIG. 4 is FIG. 3 illustrates a phonocardiogram and the corresponding beat-to-beat intervals of a five-minutes study on a subject according to the method of the present invention. The dots indicate the peaks of the heart beat automatically identified by a computer;

FIG. 5 illustrates the various frequency-domain parameters for characterizing HRV based on the analysis of information shown in FIG. 4; and

FIG. 6 shows the correlation of the various parameters in frequency domain for characterizing HRV on 10 control subjects obtained according to the method of the present invention and the conventional method.

### DESCRIPTION OF THE PREFERRED EMBODIMENTS

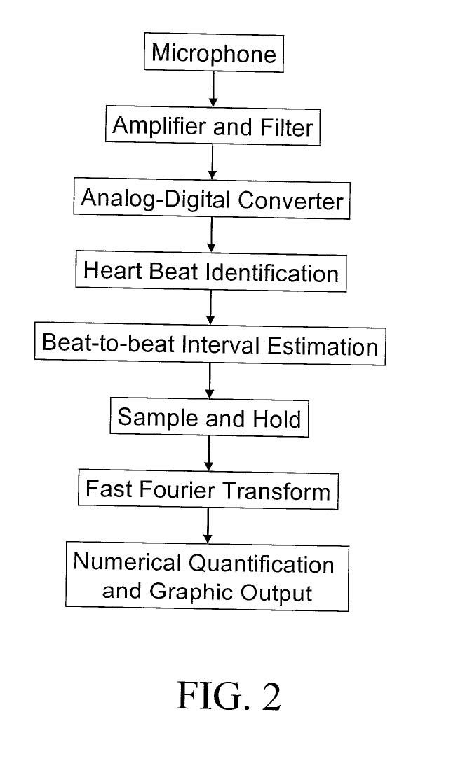

FIG. 2 is a flow diagram illustrating the approach in assessing heart rate variability (HRV) according to the present invention. The HRV of the present invention is derived from a recording of the heart sounds (phonocardiogram). As shown in FIG. 2, a microphone is used to collect a 3-minute or a 5-minute sound signals of a heart. The microphone is placed on a subject, for example, on the left chest of the subject. A hearing instrument used in auscultation can also be used to collect the sound signals of the heart. The sound signals of the heart is amplified and filtered with a band pass filter. The processed sound signals are further transmitted to an analog-to-digital (A/D) converter with a sampling rate of 1024 or 2048 Hz. The acquisition of data and the subsequent data analysis are accomplished with a computing device, which includes portable computer, personal digital assistance and microchips like those used in mobile phones and watch. The computing system must comprise also a microprocessor and adequate memory. The digitized sound signals can be analyzed on-line during a study and simultaneously stored in removal hard disks for off-line verification after the completion of the study.

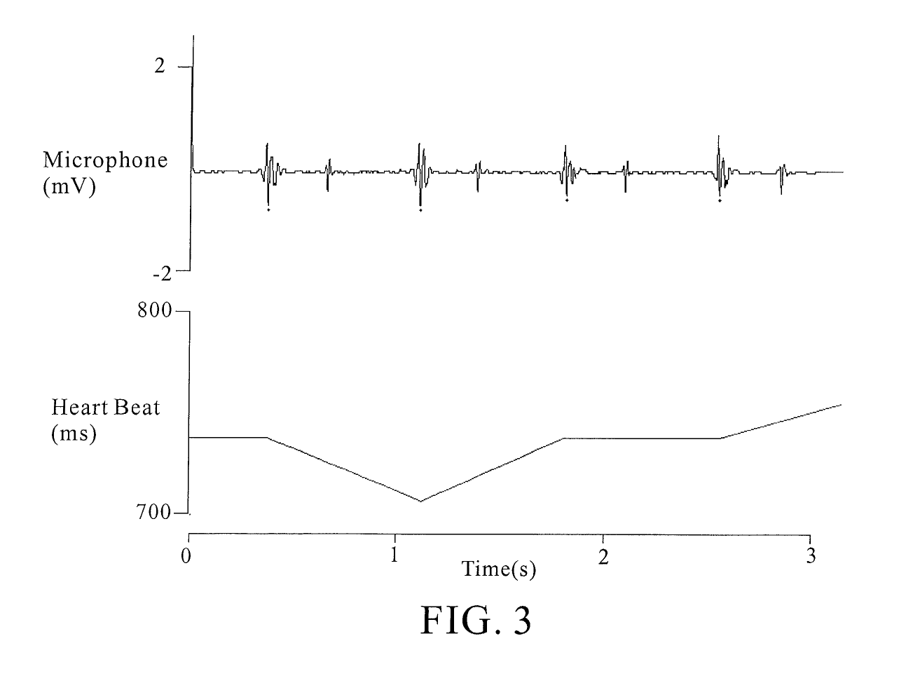

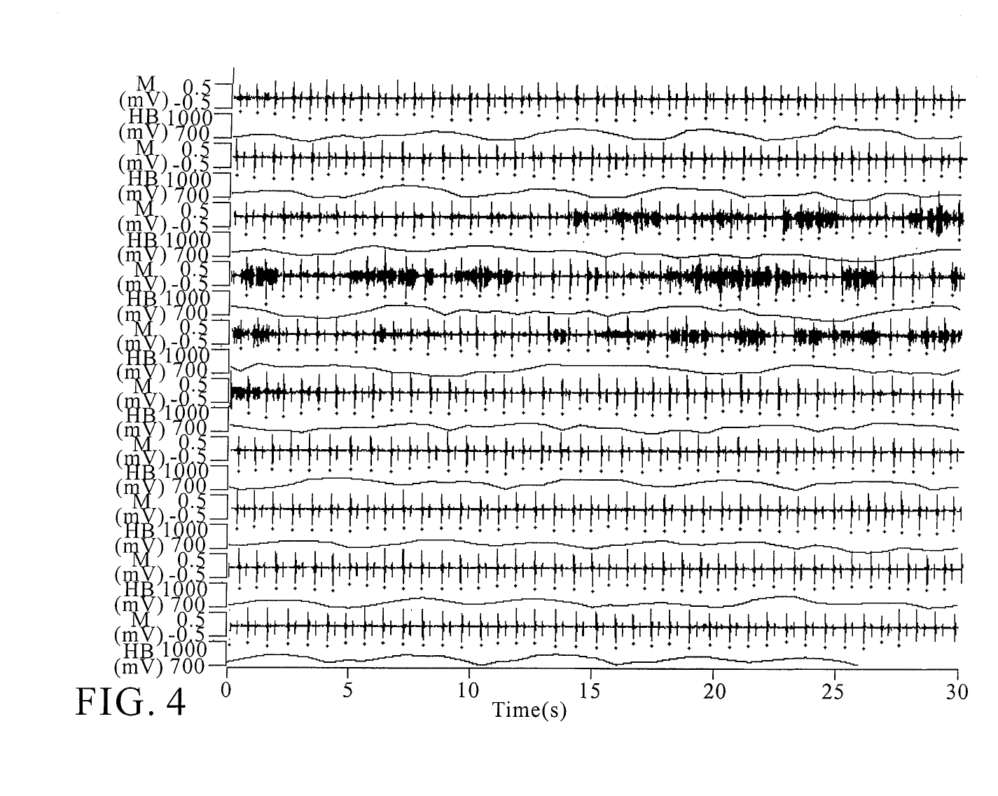

Still referring to FIG. 2, the digitized sound signals are analyzed to estimate the beat-to-beat intervals. A spike detection algorithm is used to detect all peaks of the digitized sound signals. The peak of each heart beat is defined as the time point of the heart beat, and the interval between two peaks is estimated as the beat-to-beat interval between current and latter heart beats. Parameters such as amplitude and duration of all peaks are measured so that their means and standard deviations can be calculated as standard templates. Each heart beat is then compared and validated with the standard templates. If the standard score of any of the peak interval values exceeds three, it is considered erroneous and is rejected. FIG. 3 illustrates a phonocardiogram and the corresponding beat-to-beat intervals of a five-minute study on a subject according to the method of the present invention. The dots on the phonocardiogram, which is automatically identified by the computing system, indicate the peaks of the heart beat. FIG. 4 illustrates the phonocardiograms and the corresponding beat-to-beat intervals of a five-minute study on a subject according to the method of the present invention. The dots on the phonocardiogram indicate the peaks of the heart rate automatically identified by the computing system.

Referring back to FIG. 2, the validated peak interval values are subsequently resampled and interpolated at the rate of 7.11 Hz to accomplish the continuity in time domain. Thereafter, frequency-domain analysis is performed using fast Fourier transform (FFT). The DC component of the signals is deleted, and a Hamming window is used to attenuate the leakage effect. For each 288 seconds or 2048 data points, the power spectral density is estimated on the basis of fast Fourier transform. The resulting power spectrum is corrected for attenuation resulting from the sampling and the Hamming window.

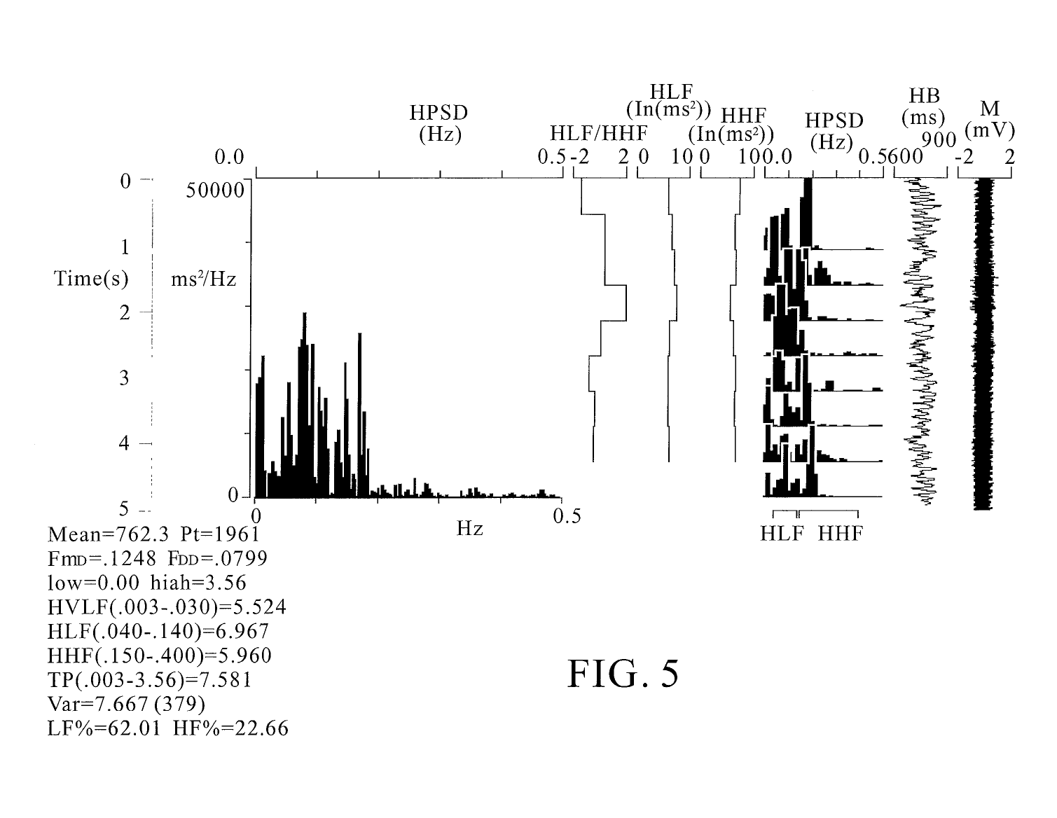

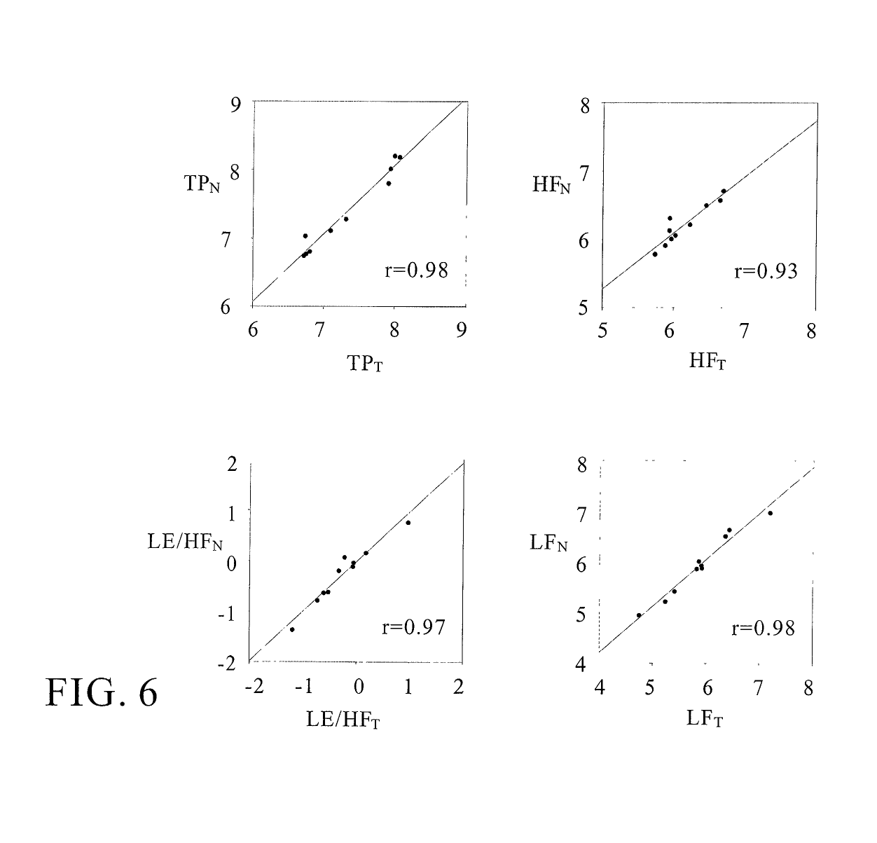

The power spectrum is subsequently quantified by means of integration into standard frequency-domain parameters including low-frequency (LF 0.04-0.15 Hz) and high-frequency (HF 0.15-0.40 Hz), total power (TP) and ratio of low frequency to high frequency (LF/HF). LF, HF, TP, and LF/HF are logarithmically transformed to correct for the skewness of distribution. FIG. 5 illustrates the various frequency-domain parameters for characterizing HRV obtained base on the analysis of information shown in FIG. 4. As shown in FIG. 5, a condensed tracing of a 5-minute phonocardiogram, the corresponding beat-to-beat intervals, power spectral density, HF, LF, BF/LF of a control subject are illustrated. FIG. 6 shows the correlation of the various parameters in frequency domain for characterizing HRV on 10 control subjects obtained according to the method of the present invention and the conventional method. All parameters exhibit good correlation with correlation coefficient (r)>0.93.

Since the HRV of the present invention is derived from a phonocardiogram, which is easily obtained by placing a microphone on a patient, the pathological conditions of a patent is readily assessable and diagnosed. Moreover, the phonocardiogram and the corresponding HRV information, even they are collected at the patient's own home, can be sent to computer systems for an off-line verification after the completion of the test. With a rapid diagnosis and transfer of information, potential consequences are mitigated and the survivability of a patient is enhanced.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. An apparatus to measure a heart rate variability (HRV), comprising:
a listening instrument to collect sound signals of a heart, wherein high frequency sounds and low frequency vibrations are transformed into electrical signals; and
a computing system to analyze the electrical signals of the sound signals of the heart, wherein frequency-domain parameters of the electrical signals are quantified to characterize the heart rate variability.

2. The apparatus of claim 1, wherein the listening instrument includes a microphone.

3. The apparatus of claim 1, wherein the listening instrument includes an instrument used in auscultation.

4. The apparatus of claim 1, wherein the apparatus further comprises an amplifier, a filter and an analog-to-digital converter to process the electrical signals before the electrical signals are analyzed by the computing system.

5. The apparatus of claim 1, wherein the computing system includes a personal computer, a personal digital assistant or a microchip.

6. The apparatus of claim 1, wherein the computing system comprises a digital signal processing unit to estimate an beat-to-beat interval of a heart beat.

7. The apparatus of claim 1, wherein the digital signal processing unit performs frequency-domain analysis, time-domain analysis and non-liner analysis to analyze the heart rate variability of the heart.

8. The apparatus of claim 6, wherein the frequency-domain parameters include high frequency (HF), low frequency (LF), total power (TP) and HF/LF.

9. A method to monitor an autonomic nervous system, comprising:
collecting sound signals of a heart resulted from contractions of the heart;
digitizing the sound signals;
estimating beat-to-beat interval values based on the digitized sound signals;
transforming the interval values into a frequency spectrum; and
quantifying components of a frequency distribution of a heart rate variability.

10. The method of claim 9, wherein the sound signals of the heart is collected by placing a microphone or a listening instrument used in auscultation near the heart of a subject.

11. The method of claim 9, wherein an interval between two peaks of a current spike and a latter spike of the digitized sound signals is estimated as the beat-to-beat value.

12. The method of claim 9, wherein estimating the beat-to-beat interval values based on the digitized sound signals further comprises:
measuring amplitudes and duration of all spikes of the digitized sound signals;
calculating means and standard deviations of the measured amplitudes and the measured duration of the spikes as standard templates;
comparing the amplitude and the duration of each spike of the digitized sound signal with the standard templates; and
rejecting the spike of the digitized sound signal if the amplitude and the duration of the spike exceeds three times of those of the standard templates.

13. The method of claim 9, wherein estimating beat-to-beat interval values, transforming the interval values into a frequency spectrum and analyzing the frequency spectrum are performed with a computer.

14. The method of claim 13, wherein the computer includes a portable computer, a personal digital assistant or a microchip.

15. The method of claim 9, wherein the components of the frequency distribution of the heart rate variability include low frequency (LF), high frequency (HF), total power (TP) and LF/HF.

16. The method of claim 9, wherein after collecting the sound signals of the heart the sound signals are amplified and filtered.
